(19) United States
(12) Patent Application Publication
(10) Pub. No.: US 2015/0051434 A1
(43) Pub. Date: Feb. 19, 2015

(54) METHOD FOR REGULARIZING APERTURE SHAPE FOR MILLING

(71) Applicant: KONINKLIJKIE PHILIPS N.V., EINDHOVEN (NL)

(72) Inventors: Prashant Kumar, Bangalore (IN); Ankit Kapoor, Bangalore (IN)

(21) Appl. No.: 14/376,962

(22) PCT Filed: Feb. 18, 2013
(86) PCT No.: PCT/IB2013/051311

§ 371 Date: Aug. 6, 2014

Related U.S. Application Data
(60) Provisional application No. 61/601,087, filed on Feb. 21, 2012.

(51) Int. Cl. A61N 5/10

(52) U.S. Cl. CPC A61N 5/1031; A61N 5/1039; A61N 2005/1096
USPC 600/1

(57) ABSTRACT

A therapy system (20) determines an aperture shape (102, 152) based on a diameter (106, 156) of a milling bit. The system (20) includes at least one processor (68, 70) programmed to receive an aperture shape (102, 152) of a treatment plan for a patient and a diameter (106, 156) of a milling bit. The aperture shape (102, 152) is regularized with respect to the diameter (106, 156) of the milling bit by at least one of over segmenting (150) a first portion of the aperture shape (102, 152) based on the diameter (106, 156) of the milling bit and under segmenting (100) a second portion of the aperture shape (102, 152) based on the diameter (106, 156) of the milling bit.

The present application relates generally to radiation therapy. It finds particular application in conjunction with radiation therapy planning and will be described with particular reference thereto. However, it is to be understood that it also finds application in other usage scenarios and is not necessarily limited to the aforementioned application.

Apertures play an important role in radiation therapy. An aperture is used to profile the beam shape with respect to the region of interest. In clinical practice, a radiation therapy planning application is used to plan the dose. Once the plan is finalized, the plan parameters are exported out for actual dose delivery, typically using Digital Imaging and Communications in Medicine (DICOM). Aperture shape is one such parameter. Typically, it is exported as a contour and used to machine a corresponding aperture. One challenge, however, is that there is often a mismatch between the exported aperture shape which is used for planning and the shape of the physical aperture that is created by the milling process.

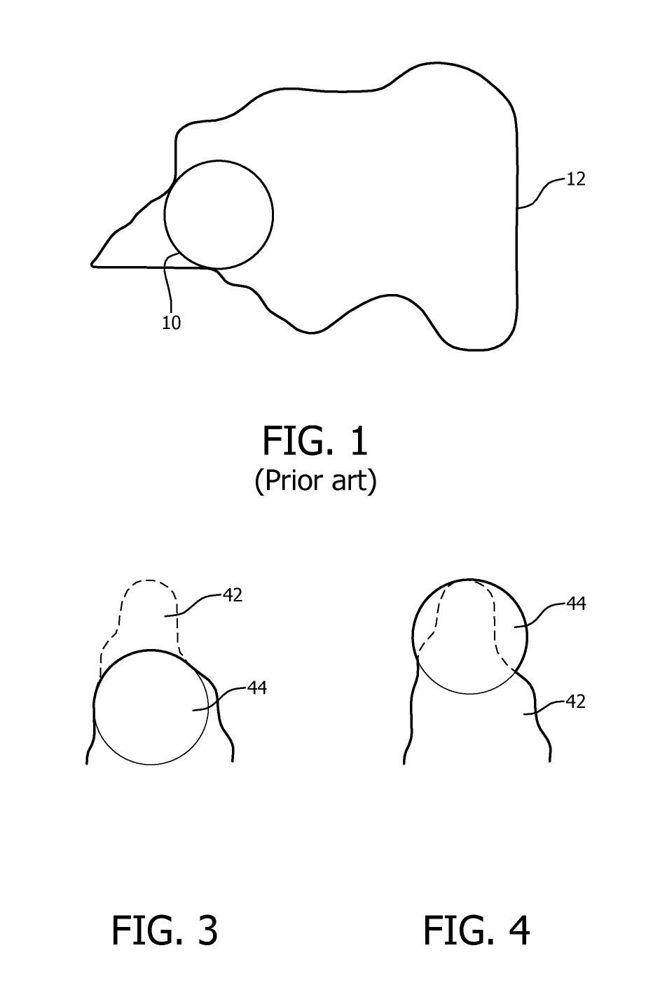

The physical aperture shape is generated by drilling an aperture block using a milling bit. If the milling bit diameter is considerably larger than the resolution of the image used for planning, such as a computed tomography (CT) image, then it is possible that the milling bit will not be able to reach the high curvature regions of the aperture shape. As a result, the milling process will fail to conform to the actual shape of the aperture. FIG. 1 illustrates a circular milling bit 10 approaching a high curvature region of an aperature shape 12, where the milling bit 10 is not able to conform to the actual shape of the aperture due to the milling bit diameter. This effect is more pronounced if the milling bit diameter is bigger and less pronounced if the milling bit diameter is smaller.

As a result of this mismatch between the exported aperture shape and the physical aperture shape, the delivered dose typically differs from the planned dose. Deviations between delivered dose and planned dose are undesirble due to, inter alia, the potential for harm to tissue and organs at risk surrounding a target and the potential for failing to adequately irradiate the target. These deviations are especially undesirable in Proton therapy, as compared to Photon therapy. Nonetheless, known radiation therapy systems do not take in to account the milling bit diameter when determining the aperture shape.

The present application provides new and improved methods and systems which overcome the above-referenced challenges and others.

In accordance with one aspect, a therapy system for determining a mask aperture shape based on a diameter of a milling bit to be used to mill an aperture is provided. The system includes at least one processor programmed to receive an aperture shape for a treatment plan for a patient and a diameter of a milling bit. The processor is further programmed to regularize the aperture shape with respect to the diameter of the milling bit.

In accordance with another aspect, a method for determining a mask aperture shape based on a diameter of a milling bit is provided. An aperture shape of a treatment plan for a patient and a diameter of a milling bit are received. The aperture shape is regularized with respect to the diameter of the milling bit.

In accordance with another aspect, a non-transitory computer readable medium carrying software which controls at least one processor to perform a method for determining a mask aperture shape based on a diameter of a milling bit is provided. The method includes receiving an aperture shape of a treatment plan for a patient and a diameter of a milling bit. The method further includes regularizing the aperture shape with respect to the diameter of the milling bit.

One advantage resides in accounting for the milling bit diameter during planning so the aperture shape can be milled.

Another advantage resides in ensuring that the planned dose and the delivered dose do not differ due to change in aperture shape.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

FIG. 1 illustrates a larger circular milling bit approaching a high curvature region of an aperature shape.

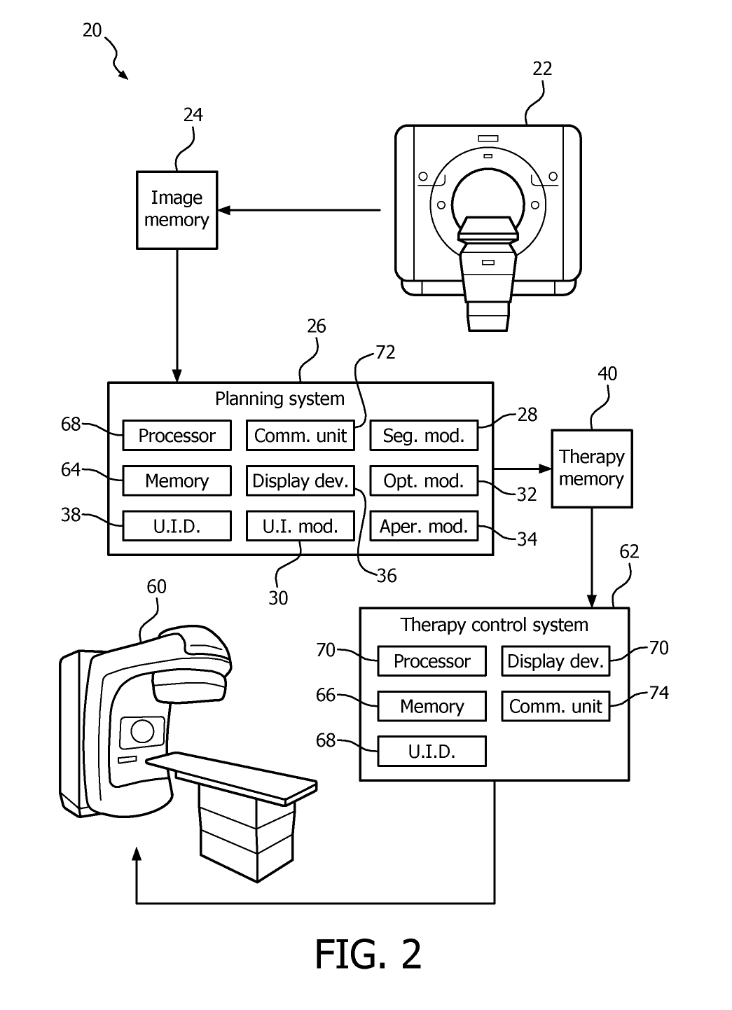

FIG. 2 illustrates a block diagram of a therapy system for determining an aperture shape based on a diameter of a milling bit.

FIG. 3 illustrates under segmentation of an aperture shape.

FIG. 4 illustrates over segmentation of an aperture shape.

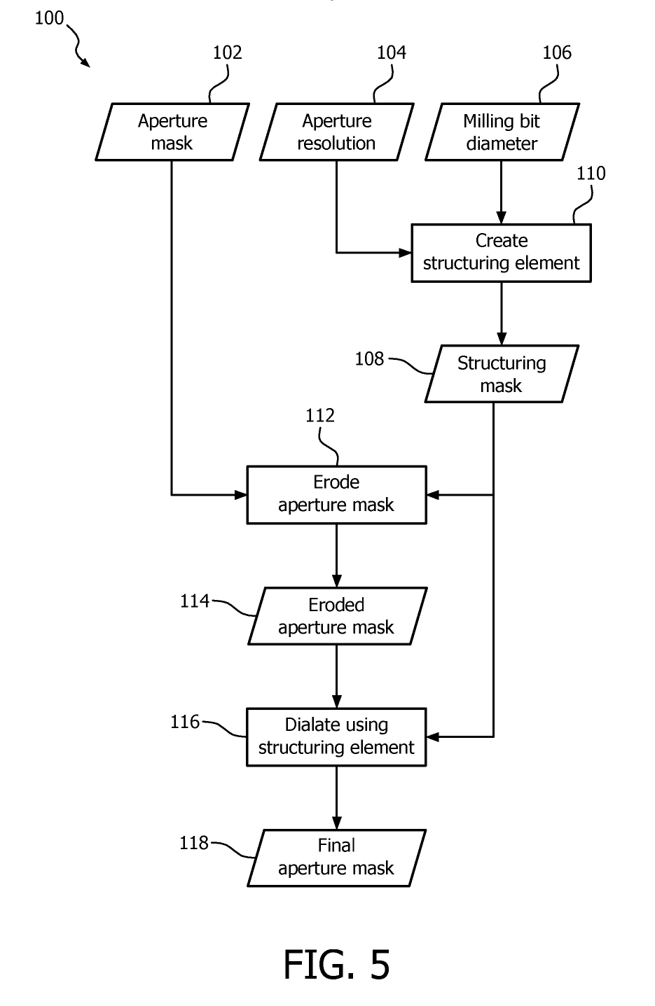

FIG. 5 illustrates a block diagram of a method for under segmenting an aperture shape.

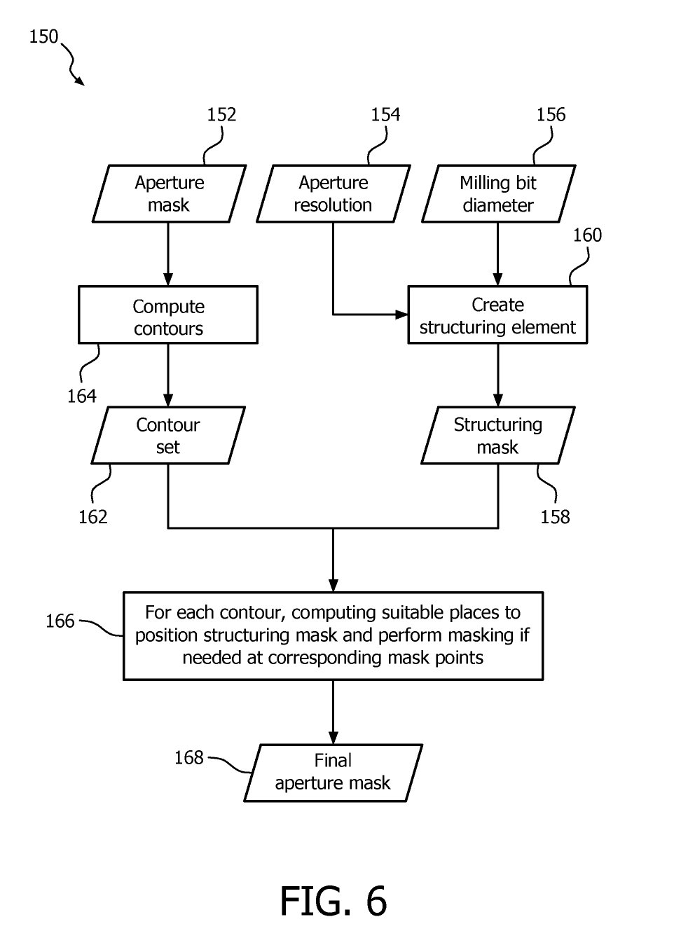

FIG. 6 illustrates a block diagram of a method for over segmenting an aperture shape.

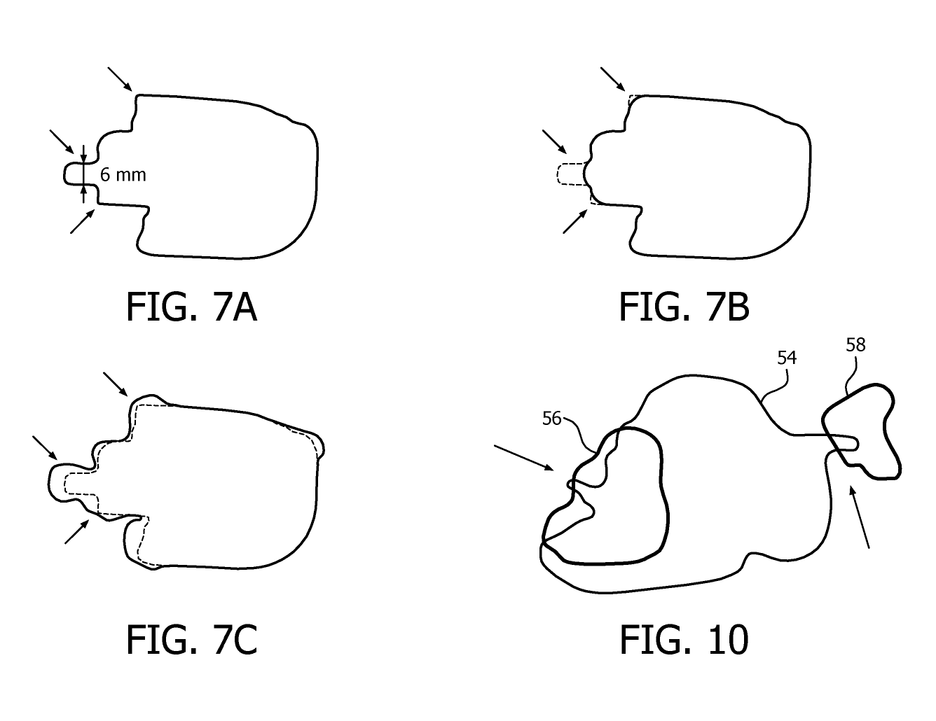

FIGS. 7A illustrates an aperture shape.

FIG. 7B illustrates an aperture shape after under segmentation.

FIG. 7C illustrates an aperture shape after over segmentation.

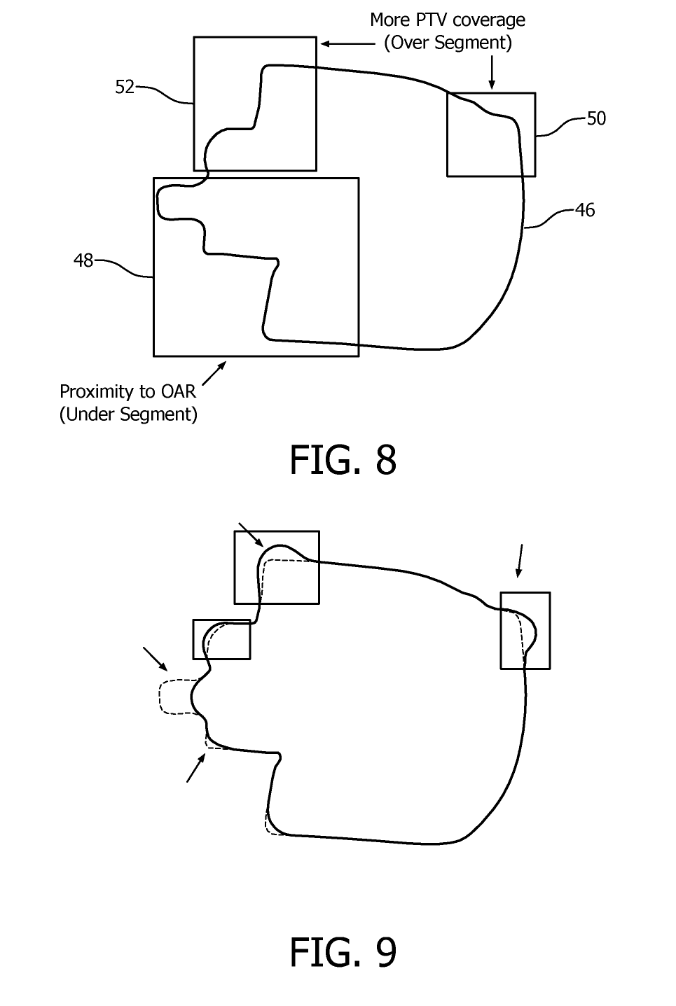

FIG. 8 illustrates selection of regions of an aperture shape for over and under segmentation.

FIG. 9 illustrates the result of over and under segmentation on different regions of an aperture shape.

FIG. 10 illustrates an aperture shape with contours for a target and organ at risk overlaid thereon.

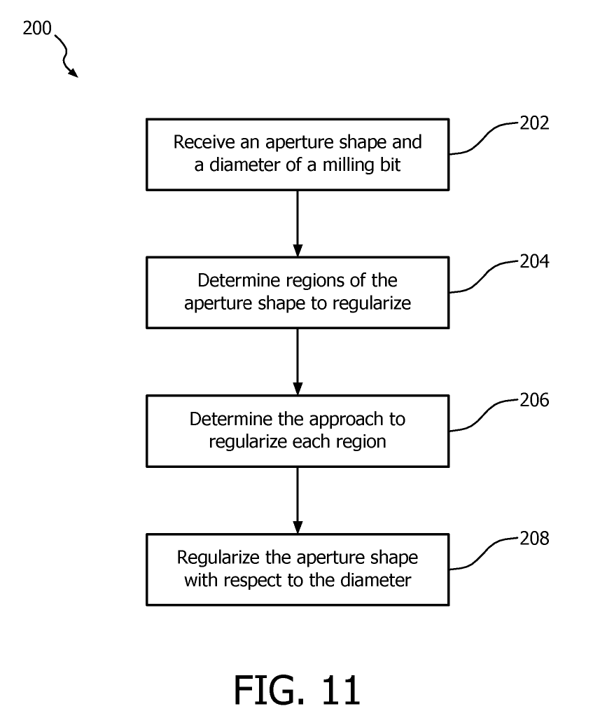

FIG. 11 illustrates a method for determining an aperture shape based on a diameter of a milling bit.

With reference to FIG. 2, a therapy system 20 includes one or more imaging modalities 22 for acquiring images of targets and/or organs at risk within patients. The imaging modalities 22 suitably include one or more of a computed tomography (CT) scanner, a positron emission tomography (PET) scanner, a magnetic resonance (MR) scanner, a single photon emission computed tomography (SPECT) scanner, a cone-beam computed tomography (CBCT) scanner, and the like. Images acquired from the imaging modalities 22 are stored in one or more image memories 24.

A therapy planning system 26 of the therapy system 20 receives a planning image, such as three- or four-dimensional image, of a target and, commonly, one or more organs at risk for a patient. The target is an organ or other tissue region which contains a lesion, such as a tumor, to be treated. Typically, the planning image is received from the imaging modalities 22 via the image memories 24, but other sources are contemplated. As discussed hereafter, the planning image is employed by the therapy planning system 26 to generate a treatment plan.

To facilitate therapy planning, the therapy planning system 26 includes one or more of a segmentation module 28, a user interface module 30, an optimization module 32, and an aperture module 34. The segmentation module 28 delineates between tissue regions, such as the target and/or the organs at risk, in the received image. Such regions are typically delineated by contours surrounding the regions. Delineation can be performed automatically and/or manually. As to the former, any number of known segmentation algorithms can be employed. As to the latter, the segmentation module 28 cooperates with the user interface module 30 to allow clinicians to manually delineate between the regions or manually adjust contours generated by an automatic segmentation algorithm.

The user interface module 30 presents a user interface to an associated user with a display device 36 of the therapy planning system 26. The user interface can allow the associated user to at least one of generate, modify and view contours. In that regard, the user interface can display the received image, a projection image, or an aperture shape, with the delineated contours optionally overlaid thereon. The associated user can then generate and/or modify contours using a user input device 38 of the therapy planning system 26. For example, a clinician can employ a mouse to resize or reshape a contour. The user interface can also allow clinicians to enter and/or define plan parameters, such as dose for contoured regions or a milling bit diameter, using the user input device 38.

The optimization module 32 receives as input at least plan parameters, such as a milling bit diameter, and contours of the target and/or the OARs, typically from the segmentation module 28 and/or the user interface module 30. The optimization module 32 optionally receives other relevant inputs, such as an attenuation map indicative of radiation absorption. Based on the inputs, the optimization module 32 generates a treatment plan complying with the plan parameters and any other relevant inputs. The treatment plan suitably includes a plurality of fractions, an aperture shape, and a planned treatment volume (PTV) to be irradiated. Treatment plans generated by the optimization module 32 are suitably stored in one or more therapy memories 40.

The optimization module 32 typically generates a treatment plan without taking in to account the milling bit diameter. To take in to account the milling bit diameter, the optimization module 32 cooperates with the aperture module 34. Namely, the optimization module 32 generates an initial treatment plan using known technqiues. This initial treatment plan fails to take in to account the milling bit diameter and includes an aperture shape. The aperture module 34 then generates a revised aperture shape taking in to account the milling bit diameter, as discussed below. Using the revised aperture shape, the optimization module 32 re-optimizes the treatment plan, optionally creating a new or modified aperture shape. This process of revising aperture shape and re-optimizing can optionally be iteratively performed a predetermined number of times.

The aperture module 34 receives an aperature shape for a treatment plan and the milling bit diameter to be employed for milling the aperature shape, typically from the optimization module 32 and/or the user interface module 30. Using the aperture shape and the milling bit diameter, the aperture module 34 regularizes the aperture shape with respect to the milling bit diameter. Discrepancy between the aperture shape generated by milling and the planned aperture shape are overcome by under segmenting the aperture shape to ensure that it can be milled and/or over segmenting the aperture shape to ensure that it can be milled.

Under segmenting entails excluding portions of the aperture shape that cannot be precisely milled due to the milling bit diameter; in other words, constricting the aperture shape to the reach of milling bit. Over segmentation entails expanding portions of the aperture shape to the milling bit diameter that could not otherwise be precisely milled due to the milling bit diameter; in other words, expanding the aperture shape with the milling bit diameter. FIGS. 3 and 4 illustrate examples of under segmenting and over segmenting a portion of an aperture shape 42, respectively, for a milling bit 44, where the darker region corresponds to the revised aperture shape. In that regard, FIG. 3 illustrates undersegmenting the aperture shape 42, where those portions of the aperture shape 42 that cannot be precisely milled by the milling bit 44 (i.e., the lighter region) are excluded from the revised aperture shape, and FIG. 4 illustrates oversegmenting the aperture shape 42, where those portions of the aperture shape 42 that cannot be precisely milled by the milling bit 44 are expanded to accommodate the milling bit 44.

With reference to FIG. 5, a block diagram of a method 100 for under segmenting an aperture shape is provided. The method 100 takes as input an aperture mask 102 (i.e., the aperture shape), an aperture resolution 104 and a milling bit diameter 106. The aperture resolution 104 is the resolution of the segmented image, such as a projection image projected parallel to a trajectory of the radiation beam. Using the aperture resolution 104 and the milling bit diameter 106, a structuring mask 108 is created 110. This entails creating a square mask with a side length equal to a corrected milling bit diameter and filling it with a circular kernel. The corrected milling bit diameter is the milling bit diameter 106 corrected for the aperture resolution 104. It is the magnification of the milling bit diameter 106 on to the patient body plane. Hence, the structuring mask 108 is representative of the circular kernel derived based on the milling bit diameter 106. Morphological opening is then employed on the aperture mask 102 using the structuring mask 108. Namely, the aperture mask 102 is eroded 112 using the structuring mask 108 to create an eroded aperture mask 114. Thereafter, the eroded aperture mask 114 is dilated 116 using the structuring mask 108 to create a final aperture mask 118.

With reference to FIG. 6, a block diagram of a method 150 for over segmenting an aperture shape is provided. As with under segmentation, the method 150 takes as input an aperture mask 152 (i.e., the aperture shape), an aperture resolution 154 and a milling bit diameter 156. The aperture resolution 154 is the resolution of the segmented image. A structuring mask 158 is then created 160 as done for under segmentation. Using these inputs, the method 150 enhances all the nooks and corners smaller than the milling bit diameter 156 to accommodate the milling bit diameter 156 corrected for image resolution.

A set of contours 162 representing the aperture shape are also computed 164 from the aperture mask 152. Thereafter, for each of the contours 162, the structuring mask 158 is moved 166 over the inside of the contour and masking is performed 166 where necessary to create a final mask 168. Namely, the structuring mask 158 is moved by moving a line segment of a predetermine length, such as the corrected milling bit diameter, joining two continuous points on the contour along the contour. The structuring mask 158 is then positioned along the normal of the line segment, the normal positioned at the point bisecting the line segment, to determine whether the aperture mask 152 needs to be altered. The aperture mask 152 needs to be altered if the bisecting point does not lie on the contour. If the aperture mask 152 needs to be altered then bitwise operations are used on the aperture mask 152 to achieve the desired aperture mask 152. The foregoing can be thought of as rolling a ball (i.e., the structuring mask 158) that moves on the inside boundary of the aperture mask 152 and checking whether the aperture mask 152 at the current location can be milled or not.

Referring back to FIG. 2, regularizing the aperture shape with respect to milling bit diameter ensures that the shape of aperture used for planning matches with the physical aperture shape generated by milling. As a result the discrepancy between the planned and delivered dose due to aperture shape mismatch is alleviated. An example of the results generated by under and over segmentation techniques are presented in FIG. 7. The three arrows highlight sections which cannot be accurately milled by a milling bit of diameter 10 mm. FIG. 7A shows the original aperture shape, FIG. 7B shows the original aperture shape after under segementation, and FIG. 7C shows the original apertuer shape after over segmentation.

While the foregoing has thus far delt with one of under segmenting and over segmenting an aperture shape, both under segmenting and over segmenting can be employed. Namely, shape regularization can be employed contextually. For example, if a region of an aperture shape that has to be regularized has proximity to an OAR, then it is better to under segment the region to spare the OAR. Likewise, if a region of an aperture shape that has to be regularized is covering the target or non-risk tissue, then it is better to over segment the region since the PTV coverage would increase.

To faciliate the use of both under segmenting and over segmenting, the aperture module 34 determines regions that have to be regularized and, for each region, applies the appropriate one of under segmentation and over segmentation. In contrast to adjusting the aperture shape using only one option (e.g., over or under segmentation), this approach gives flexibility to the associated user to selectively adjust the aperture shape for meeting the desired planning goal. With reference FIG. 8, an aperture shape 46 is provided. A rectangle 48 represents the region selected for under segmentation and a plurality of rectangles 50, 52 represent the regions marked for over segmentation. FIG. 9 shows the result of over and under segementation with the arrows pointing to regions that were regularized.

The regions to be regularized can be determined automatically and/or manually. As to the former, the regions can be determined by analyzing a difference image for differences exceeding a predetermined threshold. For example, difference of the original and an under segmented aperture mask or difference of the original and an over segmented aperture mask. As to the latter, the aperture module 34 can cooperate with the user interface module 30 to allow the associated user to select individual regions of the aperture shape. For example, the associated user can draw contours around the regions. It is also contemplated that the regions can be determined automatically and then the associated user can adjust the determined regions, as represented by contours, using the user interface.

Further, the decision as to whether to under segment or over segment a region can be determined automatically and/or manually. As to the former, the decision can be determined by computing the proximity of the region with respect to the target and/or the OARs. For example, if the region that is to be regularized with respect to milling bit diameter is close the target, over segment it, and, if the region that is to be regularized with respect to milling bit diameter is close to an OAR, under segment it. As to the latter, the aperture module 34 can cooperate with the user interface module 30 to allow the associated user to specify whether to under or over segmente the region. It is also contemplated that the decision can be made automatically and then the associated user can adjust the decision using the user interface.

With reference to FIG. 10, an aperture shape 54 is provided. Further, overlaid on the representation is a contour 56 representing a tumor and a contour 58 representing an organ at risk. The arrows point to regions that would benefit by deciding on the right regularization approach. The region close to the OAR should be under segmented and the section covering tumour should be over segmented.

Referring back to FIG. 2, at a scheduled day and time for a therapy session of a patient, a therapy delivery apparatus 60 delivers therapy to the patient. The therapy, such as ablation therapy and/or brachytherapy, can include radiation involving one or more of x-rays, gamma rays, protons, high-intensity focused ultrasound (HIFU), and the like. Suitably, the therapy delivery apparatus 60 is controlled by a therapy control system 62 in accordance with the therapy treatment plan. The therapy treatment plan can be received from, for example, the therapy memories 40. The therapy is typically irradiated with a beam directed along each of a plurality of trajectories. Difference masks can be created for different trajectories based on the projections of the target and OARs parallel to the trajectory. The radiation dose that will be delivered to the target and OARs with the determined aperture shapes is calculated. Based on the predicted delivered doses, the treatment plan can be revised. Based on the revised treatment plan, the aperture can be revised, the process can be iteratively repeated to optimze the delivered doze.

The therapy planning system 26 and the therapy control system 62 include one or more memories 64, 66 and one or more processors 68, 70. The memories 64, 66 store executable instructions for carrying out the functions associated with the therapy planning system 26 and the therapy control system 62, including those associated with the segmentation module 28, the user interface module 30, the optimization module 32, the aperture module 34. The processors 68, 70 execute the executable instructions stored on the memories 64, 66. In certain embodiments, therapy planning system 26 and/or the therapy control system 62 include communication units 72, 74 for communicating with, for example, each other, the image memories 24, the therapy memories 40, and so on, via a communications network and/or a data bus, such as a local area network or the Internet.

With reference to FIG. 11, a method 200 for determining an aperture shape based on a diameter of a milling bit is provided. The processors 68, 70 of the therapy planning system 26 and/or the therapy control system 62 suitably perform the method 200. The method 200 includes receiving 202 an aperture shape of a treatment plan for a patient and a diameter of a milling bit. The milling bit diameter is suitably the diameter of the milling bit to be used to generate the physical aperture.

Optionally, after receiving the foregoing, one or more regions of the aperture shape that cannot be milled with the milling bit are determined and/or identification of the regions is received 204. As to determining the regions, difference images can be employed, as discussed above. As to the latter, the user interface can be employed, as discussed above. Further, for each of the regions, the method 200 determines and/or receives identification 206 of whether to under segment or over segment the region. As to determining whether to under segment or over segment the region, a region proximate an organ at risk is under segmented, and a region proximate a target is over segmented.

The aperture shape is then regularized 208 with respect to the diameter of the milling bit by at least one of over segmenting a first portion of the aperture shape based on the diameter of the milling bit and under segmenting a second portion of the aperture shape based on the diameter of the milling bit. Over segmenting is performed as discussed in connection with FIG. 6 and under segmenting is performed as discussed in connection with FIG. 5. Where the regions of the aperture shape are determined and/or identification of the regions is received, the regularizing includes, for each of the regions, determining and/or receiving identification of whether to under segment or over segment the region. The region is then under segmented or over segmented according to the determination and/or the received identification.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), and the like; a user input device includes one or more of a mouse, a keyboard, a touch screen display, one or more buttons, one or more switches, one or more toggles, and the like; a database includes one or more memories; and a display device includes one or more of a LCD display, an LED display, a plasma display, a projection display, a touch screen display, and the like.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A therapy system for determining a mask aperture shape based on a diameter of a milling bit to be used to mill an aperture, said system comprising:
at least one processor programmed to:
receive an aperture shape for a treatment plan for a patient and a diameter of a milling bit; and,
regularize the aperture shape with respect to the diameter of the milling bit.

2. The therapy system according to claim 1, where the regularizing is performed by at least one of:
over segmenting a first portion of the aperture shape based on the diameter of the milling bit; and,
under segmenting a second portion of the aperture shape based on the diameter of the milling bit.

3. The therapy system according to claim 2, wherein the over segmenting includes widening the first portion of the aperture shape to at least the diameter of the milling bit.

4. The therapy system according to claim 3, wherein the over segmenting includes:
creating a structuring element representing the milling bit, the structuring element including a circular kernel sized based on the diameter;
eroding the aperture shape using the structuring element; and, dilating the eroded aperture mask fusing the structuring element.

5. The therapy system according to claim 2, wherein the under segmenting includes constricting the second portion of the aperture shape to a reach of the milling bit during milling.

6. The therapy system according to claim 5, wherein the under segmenting includes:
creating a structuring element representing the milling bit, the structuring element including a circular kernel sized based on the diameter;
moving the structuring element around an internal boundary of the aperture shape;
for each location of the structuring element as it moves around the internal boundary of the aperture shape, determine whether expansion of the aperture shape is needed at the location; and,
in response to determining expansion of the aperture shape is needed at a location, expand the aperture shape at the location.

7. The therapy system according to claim 1, wherein the processor is further programmed to:
machine an aperture according to the regularized aperture shape.

8. The therapy system (20) according to claim 1, wherein the processor is further programmed to:
re-optimize the treatment plan based on the regularized aperture shape.

9. The therapy system according to claim 8, further including:
a therapy delivery apparatus which delivers therapy to a target based on the re-optimized treatment plan.

10. The therapy system according to claim 1, wherein the over segmenting and/or the under segmenting use a corrected diameter of the milling bit, the corrected diameter determined by projecting the diameter of the milling bit onto the body of the patient using a planning image employed to generate the treatment plan.

11. The therapy system according to claim 1, wherein the processor is further programmed to:
determine and/or receive identification of one or more regions of the aperture shape that cannot be milled with the milling bit; and,
for each of the regions:
determine and/or receive identification of whether to under segment or over segment the region; and,
under segment or over segment the region according to the determination and/or the received identification.

12. The therapy system according to claim 11, wherein at least one of:
a region of the regions proximate an organ at risk is under segmented; and,
a region of the regions proximate a target is over segmented.

13. A method for determining a mask aperture shape based on a diameter of a milling bit, said method comprising:
receiving an aperture shape of a treatment plan for a patient and a diameter of a milling bit; and
regularizing the aperture shape with respect to the diameter of the milling bit.

14. The method according to claim 13, where the regularizing is performed by at least one of:
over segmenting a first portion of the aperture shape based on the diameter of the milling bit; and,
under segmenting a second portion of the aperture shape based on the diameter of the milling bit.

15. The method according to claim 14, wherein the over segmenting includes widening the first portion of the aperture shape to at least the diameter of the milling bit.

16. The method according to claim 14, wherein the under segmenting includes constricting the second portion of the aperture shape to the reach of the milling bit during milling.

17. The method according to claim 13, further including:
determining and/or receiving identification of one or more regions of the aperture shape that cannot be milled with the milling bit; and,
for each of the regions:
determining and/or receiving identification of whether to under segment or over segment the region; and,
under segmenting or over segmenting the region according to the determination and/or the received identification.

18. A non-transitory computer readable medium carrying software which controls at least one processor to perform the method according to claim 13.

19. A non transitory computer readable medium carrying software which controls at least one processor to perform a method for determining a mask aperture shape based on a diameter of a milling bit, said method comprising:
receiving an aperture shape of a treatment plan for a patient and a diameter of a milling bit; and,
regularizing the aperture shape with respect to the diameter of the milling bit.

20. The non-transitory computer readable medium according to claim 19, wherein the regularizing is performed by at least one of:
over segmenting a first portion of the aperture shape based on the diameter of the milling bit; and,
under segmenting a second portion of the aperture shape based on the diameter of the milling bit.
